(19) United States
(12) Patent Application Publication
(10) Pub. No.: US 2016/0194308 A1
(43) Pub. Date: Jul. 7, 2016

(54) NOVEL PIPERIDINYL BENZOIMIDAZOLE DERIVATIVES AS MPGES-1 INHIBITORS

(71) Applicant: NovaSAID AB, Solna (SE)

(72) Inventor: Patric STENBERG, Stockholm (SE)

(21) Appl. No.: 14/898,978

(22) PCT Filed: Jun. 20, 2013
(86) PCT No.: PCT/SE2013/050749

§ 371 Date: Dec. 16, 2015

(51) Int. Cl. C07D 405/14; A61K 31/4545

(52) U.S. Cl. CPC C07D 405/14; A61K 31/4545

(57) ABSTRACT

The compounds 1-(5-chloro-6-methyl-1-(6-(trifluoromethyl)pyridin-2-yl)-1H-benzo[d]imidazol-2-yl)-N-((3S,4R)-4-hydroxytetrahydrofuran-3-yl)piperidine-4-carboxamide and 1-(6-chloro-5-methyl-1-(6-(trifluoromethyl)pyridin-2-yl)-1H-benzo[d]imidazol-2-yl)-N-((3S,4R)-4-hydroxytetrahydrofuran-3-yl)piperidine-4-carboxamide, as well as pharmaceutically acceptable salts thereof, and a pharmaceutical composition comprising any one of these compounds. The compound are useful for the treatment and/or prevention of a disorder selected from an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia.

### FIELD OF THE INVENTION

The present invention relates to piperidinyl benzoimidazole derivatives, to their use in therapy, as well as to a pharmaceutical composition comprising said compounds.

### BACKGROUND OF THE INVENTION

Prostaglandin (PG) E₂ is produced in a sequential action including liberation of arachidonic acid, conversion into PGG₂/PGH₂ by cyclooxygenase (Cox)-1 or Cox-2 and, finally, conversion of PGH₂ into PGE₂ by prostaglandin E synthase. There exist three known enzymes that catalyze the latter reaction, i.e. microsomal prostaglandin E synthase-1 (mPGEs-1), cytosolic prostaglandin E synthase (cPGEs), and microsomal prostaglandin E synthase-2 (mPGEs-2). The latter two enzymes are constitutively expressed whereas mPGEs-1 is inducible. Initially, mPGEs-1 was regarded as the enzyme predominantly coupled with Cox-2 activity. However; later results demonstrate that mPGEs-1 can also catalyze the conversion of Cox-1 derived PGH₂ into PGE₂. mPGEs-1 possesses the highest catalytic efficiency of the known PGE synthases. The role of PGE₂ as one of the most potent mediators of inflammation together with many in vitro reports on the presence of mPGEs-1 in different models, including inflammation, suggest this enzyme to be an attractive drug target for development of new anti-inflammatory drugs with fewer side effects than the currently available NSAIDs and selective Cox-2 inhibitors [Samuelsson, B.; Morgenstern, R.; Jakobsson, P.-J. Membrane Prostaglandin E Synthase-1: A Novel Therapeutic Target. Pharmacol. Rev. 2007, 59,207-224; Jachak, S. M. PGE synthase inhibitors as an alternative to COX-2 inhibitors. Curr. Opin. Investig. Drugs, 2007, 8, 411-415].

Furthermore, mPGEs-1-derived PGE₂ has been implicated in a range of diseases, not confined to inflammation, and it is suggested that inhibitors of mPGEs-1 are effective for the treatment or prevention of these diseases also

The following published patent applications disclose certain compounds as useful in the treatment of diseases in which it is desired and/or required to inhibit the activity of a member of the MAPEG family, e.g. microsomal prostaglandin E synthase-1 (mPGEs-1):

WO/2007/042816 and WO/2008/071944 disclose certain benzoxazole derivatives.

WO/2008/084218 discloses certain benzazole derivatives

WO/2010/034797 and WO/2010/034796 disclose certain benzoimidazoles.

WO/2011/023812 discloses certain benzoimidazoles.

### SUMMARY OF THE INVENTION

One aspect of the invention is the compound 1-(5-chloro-6-methyl-1-(6-(trifluoromethyl)pyridin-2-yl)-1H-benzo[d]imidazol-2-yl)-N-((3S,4R)-4-hydroxytetrahydrofuran-3-yl)piperidine-4-carboxamide

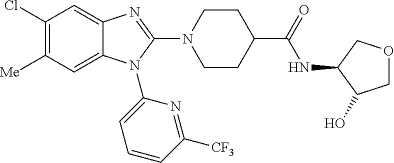

or a pharmaceutically acceptable salt thereof.

Yet an aspect of the invention is the compound (1-(6-chloro-5-methyl-1-(6-(trifluoromethyl)pyridin-2-yl)-1H-benzo[d]imidazol-2-yl)-N-((3S,4R)-4-hydroxytetrahydrofuran-3-yl)piperidine-4-carboxamide

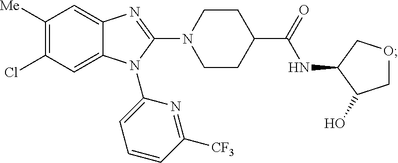

or a pharmaceutically acceptable salt thereof.

One aspect of the invention is the compound 1-(5-chloro-6-methyl-1-(6-(trifluoromethyl)pyridin-2-yl)-1H-benzo[d]imidazol-2-yl)-N-(4-hydroxytetrahydrofuran-3-yl)piperidine-4-carboxamide

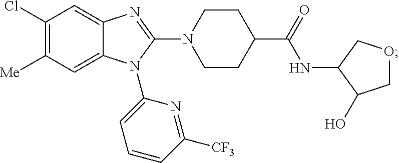 [SMILES: Cc1cc2c(cc1Cl)nc(N1CCC(C(=O)NC3COCC3O)CC1)n2-c1cccc(C(F)(F)F)n1]

or a pharmaceutically acceptable salt thereof.

Yet an aspect of the invention is the compound (1-(6-chloro-5-methyl-1-(6-(trifluoromethyl)pyridin-2-yl)-1H-benzo[d]imidazol-2-yl)-N-(4-hydroxytetrahydrofuran-3-yl)piperidine-4-carboxamide

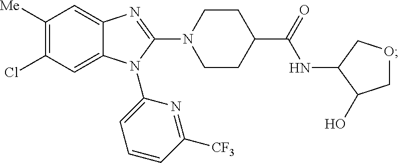 [SMILES: Cc1cc2nc(N3CCC(C(=O)NC4COCC4O)CC3)n(-c3cccc(C(F)(F)F)n3)c2cc1Cl]

or a pharmaceutically acceptable salt thereof.

A further aspect of the present invention is a compound as herein disclosed and claimed, or a pharmaceutically acceptable salt thereof, for use as a medicament.

Yet an aspect of the present invention is a pharmaceutical composition comprising a therapeutically effective amount of a compound as herein disclosed and claimed, or a pharmaceutically acceptable salt thereof, in admixture with at least one pharmaceutically acceptable excipient, e.g. an adjuvant, diluent or carrier.

An aspect of the present invention is a compound as herein disclosed and claimed for use in the treatment of a disorder in which inhibition of the activity of microsomal prostaglandin E synthase-1 is desired and/or required. Yet an aspect of the invention is the use of a compound as herein disclosed and claimed in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of an inflammatory disease, such as rheumatoid arthritis including other inflammatory diseases related to arthritis such as ankylosing spondylitis, inflammatory bowel disease, inflammation-related nephrotoxicity, osteoarthritis, periodontitis, dermatitis including psoriasis, eczema, swelling and (intra-)ocular inflammation. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of pain, such as inflammatory pain, post-operative pain, fibromyalgia, dysmenorrhea, migraine, arthrotic pain, arthritic pain, pain in connection with osteoarthritis, endometriosis, cephalalgia, pain due to gall-stones, pain due to kidney stones, pain due to gout, neuropathic pain and pain due to metastasis. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of an autoimmune disease, such as rheumatoid arthritis, multiple sclerosis and Kawasaki disease. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of a breathing disorder, such as apnea or other respiratory distress symptoms in adults and children, sudden infant death syndrome (SIDS), asthma and other chronic obstructive lung-diseases and sarcoidosis. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of cancer, such as colorectal cancer, breast cancer, gastric tumorigenesis, intestinal tumorigenesis, bone metastatic cancer, lung cancer, oesophageal adenocarcinoma, head and neck squamous cell carcinoma, papillary thyroid carcinoma, gliomas and pancreatic, cervical and prostate cancers, epithelial ovarian cancer and pancreas cancer. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of cardiovascular diseases such as myocardial infarction and stroke.

Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of a disorder selected from familial adenomatous polyposis (FAP) condition, overactive bladder, fever, and inflammation-related anorexia. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of neurodegenerative diseases, such as Alzheimer's disease, amyotrophic lateral sclerosis, memory impairment and seizure-induced neuronal damage. Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

An aspect of the present invention is a compound as herein disclosed and claimed, for use in the treatment and/or prevention of a bone disease such as bone loss or pachydermoperiostosis (PDP, also known as “primary hypertrophic osteoathropathy”). Yet an aspect of the invention is the use of a compound as herein disclosed and claimed, in the manufacture of a medicament for the treatment and/or prevention of any one of these disorders. Yet an aspect of the invention is a method for the treatment of any one of these diseases, whereby a compound as herein disclosed and claimed is administered to a subject (patient) in need of such treatment.

A further aspect of the invention is a method for the preparation of a compound as herein disclosed and claimed.

### DETAILED DESCRIPTION OF THE INVENTION

The term “treatment” as used throughout the specification and claims encompasses preventive therapy, palliative therapy or curative therapy. Thus, the term “treating” (or treatment) encompasses not only treating (or treatment of) a patient to relieve the patient of the signs and symptoms of the disease or condition, or to ameliorate the condition of the patient suffering from the disease or disorder, but also prophylactically treating an asymptomatic patient to prevent the onset or progression of the disease or condition. In one embodiment, the treatment is to relieve the patient of the signs and symptoms of the disease or condition, or to ameliorate the condition of the patient suffering from the disease or disorder or to prevent progression of the disease or condition.

The term “patient(s)” include mammalian (including human) patient(s) (or “subject(s)”).

The term “effective amount” refers to an amount of a compound that confers a therapeutic effect on the treated patient. The effect may be objective (i.e. measurable by some test or marker) or subjective (i.e. the subject gives an indication of or feels an effect).

Any chiral center in a compound of the invention having a specified configuration is indicated as R or S using the well-known Cahn-Ingold-Prelog priority rules. Also, in any structural formula a chiral center having a specified configuration, (i.e. R or S) may be indicated using

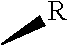

to indicate that the bond to R is directed out of the paper and towards the reader, and

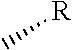

to indicate that the bond to R is directed out of the paper and away from the reader.

Compounds of the invention may be prepared according to the synthetic routes disclosed herein.

For the purpose of the present invention “pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

Examples of pharmaceutically acceptable salts useful in accordance with the invention are addition salts derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids.

Pharmaceutically acceptable excipients for use in formulating a compound according to the invention as described and claimed herein, are for example, vehicles, adjuvants, carriers or diluents, which are well-known to those skilled in the art. Pharmaceutical excipients useful in formulating a compound as herein claimed and disclosed are found in e.g. Remington: The Science and Practice of Pharmacy, 19th ed., Mack Printing Company, Easton, Pa. (1995).

Except, when otherwise indicated, e.g. by indication of (R) or (S) configuration at a given location, all stereoisomers of the compounds of the instant invention are contemplated, either in admixture or in pure or substantially pure form. Consequently, compounds of the invention may exist in enantiomeric or racemic forms or as mixtures thereof. The processes for preparation can utilize racemates or enantiomers as starting materials. When enantiomeric products are prepared, they can be separated by conventional methods, which for example are chromatographic or fractional crystallization.

The compounds of the invention can be administered for any of the uses described herein by any suitable means, for example, orally, such as in the form of tablets, aqueous or oily suspensions or solutions, elixirs, syrups, capsules, granules or powders; sublingually; buccally; ocularly; parenterally, such as by transdermal, subcutaneous, intravenous, intramuscular, or intrasternal injection or infusion techniques (e.g., as sterile injectable aqueous or non-aqueous solutions or suspensions). Also, the compounds of the invention may be applied as gargles and mouth washes. For parenteral administration, a parenterally acceptable aqueous or oily suspension, emulsion or solution is employed, which is pyrogen free and has requisite pH, isotonicity, osmolality and stability. Those skilled in the art are well able to prepare suitable formulations and numerous methods are described in the literature. A brief review of methods of drug delivery is also found in the scientific literature [eg. Langer, Science 249:1527-1533 (1990)].

Furthermore, the compounds of the invention can be administered nasally, including administration to the nasal membranes, such as by inhalation spray; topically, such as in the form of a gel, cream or ointment; or rectally such as in the form of suppositories; in dosage unit formulations containing non-toxic, pharmaceutically acceptable vehicles or diluents. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally. The precise nature of the carrier or other material will depend on the route of administration and those skilled in the art are well able to prepare suitable solutions and numerous methods are described in the literature.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of the invention can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Exemplary compositions for nasal aerosol or inhalation administration include solutions in saline which can contain, for example, benzyl alcohol or other suitable preservatives, absorption promoters to enhance bioavailability, and/or other solubilizing or dispersing agents such as those known in the art.

Exemplary compositions for parenteral administration include injectable solutions, emulsions or suspensions which can contain, for example, suitable non-toxic, parenterally acceptable diluents or solvents, such as mannitol, 1,3-butanediol, water, Ringer's solution, an isotonic sodium chloride solution, oil or other suitable dispersing or wetting and suspending agents, including synthetic mono- or diglycerides, and fatty acids, including oleic acid, or Cremaphor.

Exemplary compositions for rectal administration include suppositories which can contain, for example, a suitable non-irritating excipient, such as cocoa butter, synthetic glyceride esters or polyethylene glycols, which are solid at ordinary temperatures, but liquify and/or dissolve in the rectal cavity to release the drug.

Exemplary compositions for topical administration include a topical carrier such as Plastibase (mineral oil gelled with polyethylene).

The dose administered to a mammal, particularly a human, in the context of the present invention should be sufficient to effect a therapeutic response in the mammal over a reasonable time frame. One skilled in the art will recognize that dosage will depend upon a variety of factors including the potency of the specific compound, the age, condition and body weight of the patient, the nature and extent of the condition being treated, recommendations of the treating physician, and the therapeutics or combination of therapeutics selected for administration, as well as the stage and severity of the disease. The dose will also be determined by the route (administration form), timing and frequency of administration. Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 20 mg/kg/day, and most preferably 0.1 to 10 mg/kg/day, for adult humans. For oral administration, the compositions are preferably provided in the form of tablets or other forms of presentation provided in discrete units containing 0.5 to 1000 milligrams of the active ingredient for the symptomatic adjustment of the dosage to the patient to be treated, for example 0.5, 1.0, 2.5, 5.0, 10.0, 15.0, 25.0, 50.0, 100, 200, 400, 500, 600 and 800 mg.

Parenterally, especially intravenously, the most preferred doses will range from about 0.001 to about 10 mg/kg/hour during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art, or using so-called active transdermal technologies such as iontophoresis, electroporation, microneedles, abrasion, needle-less injection etc.

A transdermal delivery system may be continuous rather than intermittent throughout the dosage regimen. A nasal, sublingual or buccal administration may contain from about 0.01 mg to about 500 mg of a compound as herein disclosed and claimed, such as from about 1 mg to about 100 mg of active ingredient, per dose.

Compounds of the present invention may also be used or administered in combination with at least one second therapeutic agent useful in the treatment of an inflammatory disease, pain, an auto-immune disease, a breathing disorder, fever, cancer, inflammation related anorexia, Alzheimer's disease and a cardiovascular disease. The therapeutic agents may be in the same formulation or in separate formulations for administration simultaneously or sequentially. Compounds of the present invention may also be used or administered in combination with additional therapies, such as irradiation, for the treatment of cancer.

An aspect of the invention is a combination product comprising:

(A) a compound of the invention, as hereinbefore defined; and
(B) a second therapeutic agent useful in the treatment of an inflammatory disease, pain, an auto-immune disease, a breathing disorder, fever, cancer, inflammation related anorexia, Alzheimer's disease, or a cardiovascular disease, wherein each of compound (A) of the present invention, and a second therapeutic agent (B), is each formulated in admixture with a pharmaceutically acceptable excipient.

Such a combination product provides for the administration of a compound of the invention in conjunction with a second therapeutic agent, and may thus be presented either as a separate formulation, wherein at least one such formulation comprises a compound of the invention, and at least one comprises the second therapeutic agent, or may be presented (i.e. formulated) as a combined preparation (i.e. presented as a single formulation including a compound of the invention and the other therapeutic agent).

An aspect of the invention is a pharmaceutical formulation comprising a compound of the invention, as hereinbefore defined, and a second therapeutic agent, together with a pharmaceutically acceptable excipient, such as an adjuvant, diluent or carrier.

Yet an aspect of the invention is a kit of parts comprising:

(a) a pharmaceutical formulation comprising a compound of the invention, as hereinbefore defined, in admixture with a pharmaceutically acceptable excipient, such as an adjuvant, diluent or carrier; and
(b) a pharmaceutical formulation comprising a second therapeutic agent in admixture with a pharmaceutically acceptable excipient, such as an adjuvant, diluent or carrier;
wherein each component (a) and (b) are provided in a form suitable for administration in conjunction with the other.

Examples of a second therapeutic agent useful for the administration in combination with a compound of the invention, include anti-inflammatory, anti-pain, anti-autoimmune, anti-fever, anti-cancer and anti-anorexia (inflammatory) agents, and agents for the treatment or prevention of breathing disorders, cardiovascular diseases and Alzheimer's disease. a potentiator including caffeine, an H2 antagonist, aluminium or magnesium hydroxide, simethicone, a decongestant, an antitussive, an antihistamine, a diuretic, a proton pump inhibitor, a bradykinin-1 antagonist, a sodium channel blocker, a 5-HT agonist or a CGRP antagonist.

In yet an aspect of the invention, a second therapeutic agent useful for the administration in combination with a compound of the invention includes, but is not limited to, prednisone (CAS Registry Number: 53-03-2); dexamethasone (CAS Registry Number: 50-02-2); any of the selective glucocorticoid receptor agonists (SEGRAs) exemplified by A276575 (Lin, C. et al. Mol. Pharm., 2002, 62, 297-303), and ZK209614 (Schacke, H. et al, Proc. Natl. Acad. Sci. USA., 2004, 101, 227-232); aspirin (CAS Registry Number: 50-78-2); indomethacin (CAS Registry Number: 53-86-1) particularly dosed as local treatment with gel or spray; ibuprofen (CAS Registry Number: 15687-27-1); piroxicam (CAS Registry Number: 36322-90-4; CTLA4-Ig agonists/antagonists (Kremer, J. M. et al, N Engl J Med. 2003, 349, 1907-1915); Inosine-5′-monophosphate dehydrogenase inhibitors (Whitby, F. G. et al., Biochemistry, 1997, 36, 10666-10674), such as mycophenolate (CAS Registry Number: 24280-93-1); tumor necrosis factor (TNF) antagonists as infliximab (CAS Registry Number:170277-31-3); adalimumab (CAS Registry Number: 331731-18-1); etanercept (CAS Registry Number: 185243-69-0) and pentoxifylline (CAS Registry Number: 6493-05-6); orazipone (OR-1384); integrin antagonists; cell adhesion inhibitors; interferon gamma antagonists; budesonide (CAS Registry Number: 51333-22-3); clofazimine (CAS Registry Number: 2030-63-9); selective thyroid hormone agonists such as GC-1 (Johansson, L. et al., Proc. Natl. Acad. Sci. USA. 2005, 102, 10297-10302), KB2115 (Berkenstam, A. et al., Proc. Natl. Acad. Sci. USA. 2008, 105, 663-667); KB-141 (Grover, G. J. et al., Proc. Natl. Acad. Sci. USA. 2003, 100, 10067-10072); and MB07811 (Erion, M D et al., Proc. Natl. Acad. Sci. USA. 2007, 104, 15490-15495); selective farnesoid X receptor agonists (FXRs) such as WAY-362450 (Flatt, B. et al., J. Med. Chem. 2009, 52, 904-907); CD4 antagonists such as priliximab (CAS Registry Number: 147191-91-1); p38 mitogen-activated protein kinase inhibitors such as SB203580 (CAS Registry Number: 152121-47-6); mesalazine (CAS Registry Number: 89-57-6); azathioprine (CAS Registry Number: 446-86-6); sumatriptan (CAS Registry Number: 103628-46-2); paracetamol (CAS Registry Number: 103-90-2); fast-acting bronchodilators such as salbutamol (CAS Registry Number: 18559-94-9) and ephedrine (CAS Registry Number: 299-42-3); rituximab; NO-releasing drugs such as nitroglycerine (CAS Registry Number: 55-63-0) and isosorbide dinitrate (CAS Registry Number: 87-33-2); Selective Estrogen Receptor Modulators (SERMs) such as tamoxifen (CAS Registry Number:10540-29-1); raloxifene (CAS Registry Number: 84449-90-1) and toremifene (CAS Registry Number: 89778-26-7); selective Liver X receptor (LXR) agonists such as T0901317 (CAS Registry Number: 293754-55-9, Repa, J. J. et al., Science, 2000, 289, 1524-1529) and GW3965 (CAS Registry Number: 405911-17-3); anti-depressant drugs and pain-relivers, such as tricyclic antidepressants (TCAs), selective serotonin reuptake inhibitors (SSRIs) and serotonin-norepinephrine reuptake inhibitors (SNRIs) such as desvenlafaxine (CAS Registry Number: 93413-62-8); duloxetine (CAS Registry Number: 116539-59-4); milnacipran (CAS Registry Number: 92623-85-3); tramadol (CAS Registry Number: 27203-92-5) and bicifadine (CAS Registry Number: 71195-57-8).

The invention is illustrated by the following Examples:

### EXAMPLES

### Example 1

### 1-(5-chloro-6-methyl-1-(6-(trifluoromethyl)pyridin-2-yl)-1H-benzo[d]imidazol-2-yl)-N-((3S,4R)-4-hydroxytetrahydrofuran-3-yl)piperidine-4-carboxamide

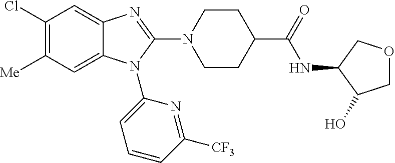

(a) Preparation of the intermediate compound 5-chloro-6-methyl-1H-benzo[d]imidazol-2(3H)-one:

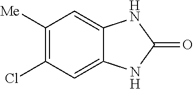 [SMILES: Cc1cc2[nH]c(=O)[nH]c2cc1Cl]

To a stirred solution of 4-chloro-5-methylbenzene-1,2-diamine (0.3 g, 1.9 mmol) in tetrahydrofuran (5 mL) was added N,N-carbonyl dimidazole (CDI, 0.465 g, 2.8 mmol). The reaction mixture was stirred at room temperature for 3 hours, concentrated in vacuo and the residue partitioned between ethyl acetate (50 mL) and aqueous sodium hydroxide (50 mL, 1 N). The aqueous extract was acidified to pH 5 via addition of aqueous hydrochloric acid (1.5 N) and the resulting brown precipitate was filtered, washed with water (3×20 mL) and dried under high vacuum to obtain 0.28 g (80% yield) of 5-chloro-6-methyl-1H-benzo[d]-imidazol-2(3H)-one.

(b) Preparation of the intermediary tautomeric compounds 2,6-dichloro-5-methyl-1H-benzo[d]-imidazole hydrochloride and 2,5-dichloro-6-methyl-1H-benzo[d]imidazole hydrochloride:

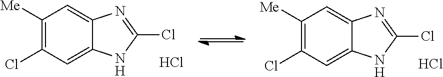 [SMILES: Cc1cc2nc(Cl)[nH]c2cc1Cl ; Cl ; Cc1cc2nc(Cl)[nH]c2cc1Cl ; Cl]

To 5-chloro-6-methyl-1H-benzo[d]imidazol-2(3H)-one (0.627 g, 3.43 mmol), phosphoryl trichloride (POCl₃, 21.1 g, 0.137 mol) was added, the reaction mixture was heated at 100° C. for 6 hours and subsequently cooled down to room temperature. The formed precipitate was filtered off and washed with toluene. The filtrate was concentrated in vacuo and collected. This gave 674 mg (83% yield) of 2,5-dichloro-6-methyl-1H-benzo[d]imidazole hydrochloride and 2,6-dichloro-5-methyl-1H-benzo[d]imidazole hydrochloride. LC-MS (m/z) 200.9 (M+1). No effort was done to determine the ratio of the tautomers.

(c) Preparation of the intermediary tautomeric compounds ethyl 1-(5-chloro-6-methyl-1H-benzo[d]imidazol-2-yl)piperidine-4-carboxylate and ethyl 1-(6-chloro-5-methyl-1H-benzo[d]imidazol-2-yl)piperidine-4-carboxylate:

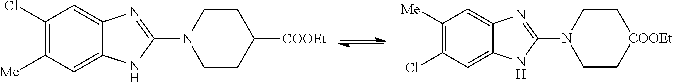

2,5-dichloro-6-methyl-1H-benzo[d]imidazole hydrochloride and 2,6-dichloro-5-methyl-1H-benzo[d]imidazole hydrochloride (0.622 g, 3.094 mmol), 1,4-dioxane (4 mL), N,N-diisopropylethylamine (Hünig's base, DIEA, 0.400 g, 3.094 mmol) and ethyl piperidine-4-carboxylate (0.584 g, 3.712 mmol) was subjected to microwave conditions for one hour at 180° C. The reaction mixture was concentrated in vacuo and purified on column (silica gel using an automated flash chromathography Biotage SP1, iso-hexane/ethyl acetate, gradient elution using 25 to 100% ethyl acetate) to give 0.617 g (62% yield) of ethyl 1-(5-chloro-6-methyl-1H-benzo-[d]imidazol-2-yl)piperidine-4-carboxylate and ethyl 1-(6-chloro-5-methyl-1H-benzo[c/]-imidaz-ol-2-yl)piperidine-4-carboxylate. No effort was done to determine the ratio of the tautomers. LC-MS (m/z) 322.2 (M+1).

(d) Preparation of the intermediary intermediate compounds ethyl 1-(5-chloro-6-methyl-1-(6-(trifluoromethyl)pyridin-2-yl)-1H-benzo[d]imidazol-2-yl)piperidine-4-carboxylate and ethyl 1-(6-chloro-5-methyl-1-(6-(trifluoromethyl)pyridin-2-yl)-1H-benzo[d]imidazol-2-yl)piperidine-4-carboxylate:

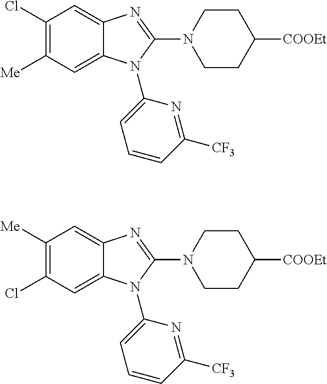 [SMILES: CCOC(=O)C1CCN(c2nc3cc(Cl)c(C)cc3n2-c2cccc(C(F)(F)F)n2)CC1 ; CCOC(=O)C1CCN(c2nc3cc(C)c(Cl)cc3n2-c2cccc(C(F)(F)F)n2)CC1]

A tautomeric mixture of ethyl 1-(5-chloro-6-methyl-1H-benzo[d]imidazol-2-yl)piperidine-4-carboxylate and ethyl 1-(6-chloro-5-methyl-1H-benzo[d]-imidaz-ol-2-yl)piperidine-4-carboxylate (100 mg, 0.311 mmol), acetonitrile (0.8 mL), caesium carbonate (142 mg, 0.435 mmol), 2-bromo-6-(trifluoromethyl)pyridine (211 mg, 0.932 mmol), 8-hydroxyquinoline (9.02 mg, 0.0621 mmol), polyethylene glycol 400 (62.2 mg, 0.155 mmol) and copper(I) oxide (8.89 mg, 0.0621 mmol) was subjected to microwave conditions for one hour at 160° C. The reaction mixture concentrated in vacuo and purified on column (silica gel using an automated flash chromate-graphy Biotage SP1, iso-hexane/ethyl acetate, gradient elution using 20 to 40% ethyl acetate). This gave 44.3 mg (31% yield) of ethyl 1-(5-chloro-6-methyl-1-(6-(trifluoromethyl)-pyridin-2-yl)-1H-benzo[d]-imidazol-2-yl)piperidine-4-carboxylate and 41.6 mg (29% yield) of ethyl 1-(6-chloro-5-methyl-1-(6-(trifluoromethyl)pyridin-2-yl)-1H-benzo[d]-imidazol-2-yl)piperidine-4-carboxylate, both as white solids and displaying LC-MS (m/z) of 467.14 and 467.13 (M+1), respectively.

(e) Ethyl 1-(5-chloro-6-methyl-1-(6-(trifluoromethyl)-pyridin-2-yl)-1H-benzo[d]imidazol-2-yl)-piperidine-4-carboxylate (20.6 mg, 0.0441 mmol) was dissolved in ethanol (1.0 mL) and aqueous sodium hydroxide (1 N, 0.221 mL) was added. The reaction mixture was stirred at room temperature for one hour, neutralized with aqueous hydrochloric acid (2 N) and evaporated to dryness. The residue was mixed with (3R,4S)-4-aminotetrahydrofuran-3-ol (5.5 mg, 0.053 mmol), N,N-diisopropylethylamine (Hünig's base, DIEA, 17.1 mg, 0.133 mmol) and N,N-dimethylformamide (1.0 mL). 2-(7-Aza-1H-benzotriazole-1-yl)-1,1,3,3-tetramethyluronium hexafluorophosphate (HATU, 18.5 mg, 0.049 mmol) was added, the reaction mixture stirred for one hour and concentrated in vacuo. The residue was purified on column (silica gel using an automated flash chromatography Biotage SP1, ethyl acetate/methanol, gradient elution using 4 to 10% methanol) to give 19.7 mg (85% yield) of 1-(5-chloro-6-methyl-1-(6-(trifluoromethyl)-pyridin-2-yl)-1H-benzo[d]imidazol-2-yl)-N-((3S,4R)-4-hydroxytetrahydrofuran-3-yl)piperidine-4-carboxamide. LC-MS (m/z) 524.14 (M+1).

### Example 2

### 1-(6-chloro-5-methyl-1-(6-(trifluoromethyl)pyridin-2-yl)-1H-benzo[d]imidazol-2-yl)-N-((3S,4R)-4-hydroxytetrahydrofuran-3-yl)piperidine-4-carboxamide

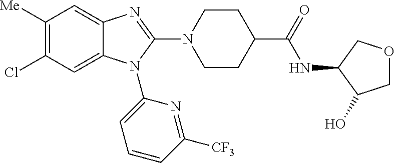

Ethyl 1-(6-chloro-5-methyl-1-(6-(trifluoromethyl)-pyridin-2-yl)-1H-benzo[d]imidazol-2-yl)-piperidine-4-carboxylate (21 mg, 0.045 mmol) was dissolved in ethanol (1.0 mL) and aqueous sodium hydroxide (1 N, 0.225 mL) was added. The reaction mixture was stirred at room temperature for one hour, neutralized with aqueous hydrochloric acid (2 N) and evaporated to dryness. The residue was mixed with (3R,4S)-4-aminotetrahydrofuran-3-ol (5.6 mg, 0.054 mmol), N,N-diisopropylethylamine (Hünig's base, DIEA, 17.4 mg, 0.135 mmol) and N,N-dimethylformamide (1.0 mL). 2-(7-Aza-1H-benzotriazole-1-yl)-1,1,3,3-tetramethyluronium hexafluorophosphate (HATU, 18.76 mg, 0.049 mmol) was added, the reaction mixture stirred for one hour and concentrated in vacuo. The residue was purified on column (silica gel using an automated flash chromatography Biotage SP1, ethyl acetate/methanol, gradient elution using 4 to 10% methanol) to give 18.8 mg (80% yield) of 1-(6-chloro-5-methyl-1-(6-(trifluoromethyl)-pyridin-2-yl)-1H-benzo[d]imidazol-2-yl)-N-((3S,4R)-4-hydroxytetrahydrofuran-3-yl)piperidine-4-carboxamide. LC-MS (m/z) 524.28 (M+1).

### Comparative Example 3 (not According to the Invention)

### (R)-1-(5-chloro-6-methyl-1-(5-(trifluoromethyl)pyridin-2-yl)-1H-benzo[d]imidazol-2-yl)-N-(tetrahydrofuran-3-yl)-piperidine-4-carboxamide

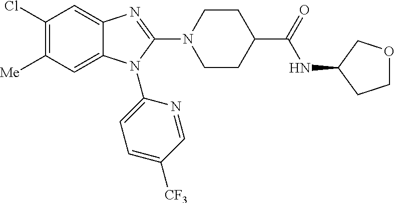 [SMILES: Cc1cc2c(cc1Cl)nc(N1CCC(C(=O)N[C@@H]3CCOC3)CC1)n2-c1ccc(C(F)(F)F)cn1]

This compound was prepared as described in Example 272 of the International application No. WO2011/023812, of NovaSAID AB.

### Comparative Example 4 (not According to the Invention)

### (R)-1-(6-chloro-5-methyl-1-(5-(trifluoromethyl)pyridin-2-yl)-1H-benzo[d]imidazol-2-yl)-N-(tetrahydrofuran-3-yl)-piperidine-4-carboxamide

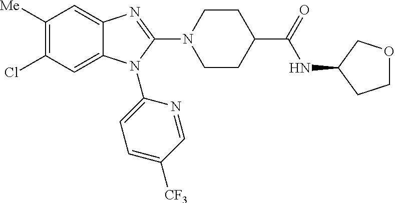 [SMILES: Cc1cc2nc(N3CCC(C(=O)N[C@@H]4CCOC4)CC3)n(-c3ccc(C(F)(F)F)cn3)c2cc1Cl]

This compound was prepared as described in Example 271 of the International application No. WO2011/023812, of NovaSAID AB.

### Comparative Example 5 (not According to the Invention)

### (R)-1-(5,6-dichloro-1-(6-(trifluoromethyl)pyridin-2-yl)-1H-benzo[d]imidazol-2-yl)-N-(tetrahydrofuran-3-yl)piperidine-4-carboxamide

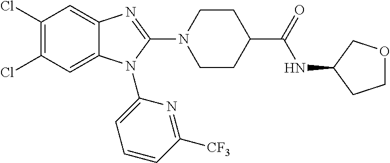 [SMILES: O=C(N[C@@H]1CCOC1)C1CCN(c2nc3cc(Cl)c(Cl)cc3n2-c2cccc(C(F)(F)F)n2)CC1]

This compound was prepared as described in Example 221 of the International application No. WO2011/023812, of NovaSAID AB.

### Biological Tests

### Thiobarbituric Acid Assay (TBA-MDA Assay or Malondialdehyde Assay)

Malondialdehyde is a product of lipid peroxidation and reacts with thiobarbituric acid forming a red product that absorbs light at 535 nm (W. G. Niehaus, Jr and B. Samuelsson, Eur. J. Biochem 6, 126 (1968)) and is also fluorescent. The extinction coefficient of the TBA-MDA conjugate is 1.56×10⁵ M⁻¹ cm⁻¹ (E. D. Wills. Biochem. J. 113, 315 (1969). The method used for detection of inhibition of mPGEs-1 is based on the detection of the amount of remaining PGH₂. This method was described more than 20 years ago by Basevich et al (Bioorg. Khim. 1983, 9(5), 658-665).

The assay used was a modified variant and used citric acid instead of the TCA-TBA-HCl reagent described in the original assay. In this assay, recombinant membrane-bound human mPGEs-1 was incubated with PGH₂. The reaction was stopped by adding citric acid to a final pH 3 and a large excess of iron(II) chloride (20 mM) to convert any remaining PGH₂ into MDA and 12-HHT. TBA reagent was finally added (0.67%) and the samples were heated at 80° C. for 30 min. The fluorescence of the conjugate was measured at 485/545 nm.

The product of mPGEs-1 (PGE₂) was not measured directly in this assay, but rather the remaining substrate (PGH₂) indirectly by adding FeCl₂ that converts PGH₂ into MDA and 12-HHT. As a positive control, a known mPGEs-1 inhibitor, MK-886, was used and the inhibition afforded by the new inhibitors was compared with that obtained from MK-886.

Automated Whole-Cell Patch-Clamp hERG Assay

The automated whole-cell patch-clamp hERG assay uses CHO-K1 cells obtained from American Tissue Culture Collection. hERG cDNA (GenBank sequence NM_000238) was subcloned into pSI vector (Promega). The CHO-K1 cells were co-transfected with this construct and pPUR (containing puromycin selective marker, BD Bioscience). After selection in puromycin for 10 days, single colonies were selected and verified with hERG potassium currents. The stably transfected cells were cultured in F-12 Kaighn's Nutrient Mixture medium (Invitrogen)+10% FBS at 37 C for 1-3 days. Subsequently the cells were harvested by trypsination and kept in serum free medium at room temperature before recording. The cells were washed and resuspended in extracellular solution before being applied to the patch clamp sites.

After whole cell configuration was achieved, the cell was held at −80 mV. A 50 millisecond pulse to −40 mV was delivered to measure the leaking current, which was subtracted from the tail current on-line. Then the cell was de-polarized to +20 mV for 2 seconds, followed by a second pulse to −40 mV to reveal hERG tail current. The paradigm was delivered once every 5 seconds to monitor the current amplitude. The extracellular solution (control) was applied first and the cell was stabilized in extracellular solution for 5 minutes. Then the test compound was applied from low concentrations (0.39 μM) to high concentrations (100 μM) cumulatively. The cell was incubated with each test concentration for 5 minutes. During the incubation, the cell was repeatedly stimulated using the voltage protocol described above, and the tail current amplitude was continuously monitored.

Data were acquired by Qpatch (Sophion Bioscience) and the degree of inhibition (%) was obtained by measuring the tail current amplitude before and after test compound incubation (the difference in current was normalized to control and multiplied by 100 to obtain the percent of inhibition). Concentration (log) response curves were fitted to a logistic equation (three parameters assuming complete block of the current at very high test compound concentrations) to generate estimates of the 50% inhibitory concentration (IC50). The concentration-response relationship of each compound was constructed from the percentage reductions of current amplitude by sequential concentrations (0.39 μM to 100 μM).

Further background information on the hERG assay is available in Expert Opin. Ther. Targets (2006) 10(2):319-327.

The arithmetic mean values from the human mPGEs-1 TBA-MDA assay (IC50 in μM) are shown in Table 1, which also shows the hERG assay IC50 values (in μM) obtained for the inventive compounds of Example 1 and 2, and the prior art compounds of Examples 3-5, respectively.

TABLE 1

| | Human mPGEs-1 | hERG | IC50 ratio |
|---|---|---|---|
| Compound | IC50 (μM) | IC50 (μM) | hERG/mPGEs-1 |
| Example 1 | 0.038 | 38 | 1000 |
| Example 2 | 0.032 | >100 | >3125 |
| Comparative | 0.058 | 19 | 328 |
| Example 3 | | | |
| Comparative | 0.046 | 14 | 304 |
| Example 4 | | | |
| Comparative | 0.024 | 2.1 | 88 |
| Example 5 | | | |

The experimental results represented in Table 1 support that compounds of the present invention are capable of inhibiting human mPGEs-1 while having an advantageous hERG/mPGEs-1 IC50 ratio, compared to the prior art compounds.

1. The compound 1-(5-chloro-6-methyl-1-(6-(trifluoromethyl)pyridin-2-yl)-1 H-benzo[d]imidazol-2-yl)-N-((3S,4R)-4-hydroxytetrahydrofuran-3-yl)piperidine-4-carboxamide 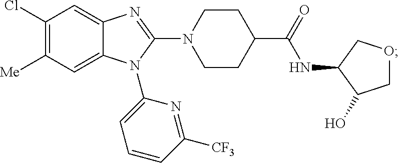
or a pharmaceutically acceptable salt thereof.

2. The compound 1-(6-chloro-5-methyl-1-(6-(trifluoromethyl)pyridin-2-yl)-1 H-benzo[d]imidazol-2-yl)-N-((3S,4R)-4-hydroxytetrahydrofuran-3-yl)piperidine-4-carboxamide 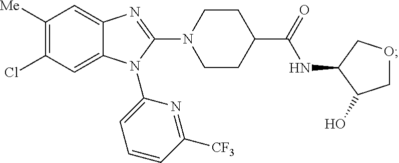
or a pharmaceutically acceptable salt thereof

3. The compound according to claim 1, or a pharmaceutically acceptable salt thereof, for use as a medicament.

4. A pharmaceutical composition comprising a therapeutically effective amount of a compound according to claim 1, or a pharmaceutically acceptable salt thereof, in admixture with at least one pharmaceutically acceptable excipient or carrier.

5. A combination product comprising: (A) a compound according to claim 1, or a pharmaceutically acceptable salt thereof; and (B) a second therapeutic agent.

6. The compound according to claim 1, or a pharmaceutically acceptable salt thereof, for use in the treatment and/or prevention of a disorder selected from:
an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia.

7. The use of a compound according to claim 1, or a pharmaceutically acceptable salt thereof, in the manufacture of a medicament for the treatment and/or prevention of a disorder selected from:
an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia.

8. A method for the treatment and/or prevention of a disorder selected from:
an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia,
whereby a compound according to claim 1, or a pharmaceutically acceptable salt thereof, is administered to a subject in need of such treatment.

9. The compound according to claim 2, or a pharmaceutically acceptable salt thereof, for use as a medicament.

10. A pharmaceutical composition comprising a therapeutically effective amount of a compound according to claim 2, or a pharmaceutically acceptable salt thereof, in admixture with at least one pharmaceutically acceptable excipient or carrier.

11. A combination product comprising: (A) a compound according to claim 2, or a pharmaceutically acceptable salt thereof; and (B) a second therapeutic agent.

12. The compound according to claim 2, or a pharmaceutically acceptable salt thereof, for use in the treatment and/or prevention of a disorder selected from:
an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia.

13. The use of a compound according to claim 2, or a pharmaceutically acceptable salt thereof, in the manufacture of a medicament for the treatment and/or prevention of a disorder selected from:
an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia.

14. A method for the treatment and/or prevention of a disorder selected from:
an inflammatory disease; an autoimmune disease; pain; a breathing disorder; cancer; a cardiovascular disease; a neurodegenerative disease; a bone disease; a disorder due to familial adenomatous polyposis (FAP) condition; overactive bladder; fever; and inflammation-related anorexia,
whereby a compound according to claim 2, or a pharmaceutically acceptable salt thereof, is administered to a subject in need of such treatment.
